(19) United States
(12) Patent Application Publication
(10) Pub. No.: US 2023/0377068 A1
(43) Pub. Date: Nov. 23, 2023

(54) METHODS AND SYSTEMS FOR SOCIAL ASSISTANCE REVIEW OF SMART CITY BASED ON INTERNET OF THINGS

(71) Applicant: CHENGDU QINCHUAN IOT TECHNOLOGY CO., LTD., Chengdu (CN)

(72) Inventors: Zehua SHAO, Chengdu (CN); Haitang XIANG, Chengdu (CN); Yaqiang QUAN, Chengdu (CN); Yong LI, Chengdu (CN); Xiaojun WEI, Chengdu (CN)

(73) Assignee: CHENGDU QINCHUAN IOT TECHNOLOGY CO., LTD., Chengdu (CN)

(21) Appl. No.: 17/810,822

(22) Filed: Jul. 5, 2022

(30) Foreign Application Priority Data
May 18, 2022 (CN) 202210538574.9

(51) Int. Cl. G06Q 50/10; G06Q 20/40; G16H 10/60

(52) U.S. Cl. CPC G06Q 50/10; G06Q 20/4016; G16H 10/60

(57) ABSTRACT

The embodiments of the present disclosure provide a method for social assistance review of a smart city based on Internet of Things, the method comprises obtaining a social assistance application of an assistance applicant based on a user platform by a social assistance service platform; obtaining auxiliary reviewing information; and determining an assistance object passing the review by reviewing the social assistance application based on the auxiliary reviewing information.

### CROSS-REFERENCE TO RELATED APPLICATIONS

This application claims priority to Chinese Patent Application No. 202210538574.9, filed on May 18, 2022, the contents of which are hereby incorporated by reference to its entirety.

### TECHNICAL FIELD

The present disclosure relates to a field of Internet of Things and social assistance, and specifically relates to methods and systems for social assistance review of a smart city based on Internet of Things.

### BACKGROUND

Although the current society has been developing at a high speed, many poor people still need to be assisted by society. Some non-poor people use false information to apply for social assistance through certain means, which makes social assistance resources face a risk that people real poor are not helped.

The Internet of Things is an important part of the new generation of information technology and is an extension and expansion network based on the Internet. A huge network is formed by combining various information sensing devices with the network to realize the interconnection of people, machines, and things at any time and any place. This provides a technical foundation for cities with dense population and different levels of rich and poor of population. It is of great significance to design a system for social assistance review of a smart city based on Internet of Things through the Internet of Things technology.

Therefore, a method for social assistance review of a smart city based on Internet of Things is required, which can accurately identify personnel who really need social assistance.

### SUMMARY

One of the embodiments of the present disclosure provides a method for social assistance review of a smart city based on Internet of Things. The method includes: obtaining a social assistance application of an assistance applicant based on a user platform by a social assistance service platform; obtaining auxiliary reviewing information;

and determining an assistance object passing the review by reviewing the social assistance application based on the auxiliary reviewing information.

One of the embodiments of the present disclosure provides a system for social assistance review of a smart city based on Internet of Things, comprising a user platform, a social assistance service platform, and a social assistance management platform. The social assistance management platform is configured to perform the following operations including: obtaining a social assistance application of an assistance applicant based on the user platform by the social assistance service platform; obtaining auxiliary reviewing information; and determining an assistance object passing the review by reviewing the social assistance application based on the auxiliary reviewing information.

One of the embodiments of the present disclosure provides a computer-readable storage medium comprising computer instructions, wherein when reading the computer instructions in the storage medium, the computer executes the method for social assistance review of the smart city based on Internet of Things.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which the same reference numerals represent the same structures, and wherein:

FIG. 1 is a schematic diagram of the application scenario of a system for social assistance review of a smart city based on Internet of Things according to some embodiments of the present disclosure;

FIG. 2 is a schematic diagram illustrating an exemplary process for social assistance review of a smart city based on Internet of Things according to some embodiments of the present disclosure;

FIG. 3 is a flowchart illustrating an exemplary process for social assistance review of a smart city based on Internet of Things according to some embodiments of the present disclosure;

FIG. 4 is a flowchart illustrating an exemplary process for obtaining auxiliary reviewing information according to some embodiments of the present disclosure;

FIG. 5 is a flowchart illustrating an exemplary process for obtaining auxiliary reviewing information according to the other embodiments of the present disclosure;

FIG. 6 is a flowchart illustrating an exemplary process for obtaining fraud risk information according to some embodiments of the present disclosure.

### DETAILED DESCRIPTION

In order to more clearly illustrate the technical solutions of the embodiments of the present disclosure, the following will briefly introduce the drawings that need to be used in the description of the embodiments. Obviously, the drawings in the following description are only some examples or embodiments of the present disclosure, and those skilled in the art may apply this present disclosure to other similar situations based on these drawings and on the premise of not paying creative labor. Unless obviously obtained from the context or the context illustrates otherwise, the same numeral in the drawings refers to the same structure or operation.

It should be understood that “systems”, “devices”, “units”, and/or “modules” used herein are one method for distinguishing different components, elements, components, parts, or assemblies of different levels. However, if other words may achieve the same purpose, the words may be replaced by other expressions.

As shown in the present disclosure and the claims, unless the context clearly suggests exceptional circumstances, the words “a”, “an” and/or “the” do not specifically refer to the singular forms, but may also include the plural forms; and the plural forms may be intended to include singular forms as well. In general, the terms “comprise,” “comprises,” and/or “comprising,” “include,” “includes,” and/or “including,” merely prompt to include steps and elements that have been clearly identified, and these steps and elements do not constitute an exclusive listing. The methods or devices may also include other steps or elements.

The flowcharts used in the present disclosure illustrate operations that the system implements according to some embodiments of the present disclosure. It should be understood that the previous or back operations may not be accurately implemented in order. Conversely, the operations may be implemented in an inverted order, or simultaneously. At the same time, other operations may also be added to these processes, or a certain step or several steps may be removed from these processes.

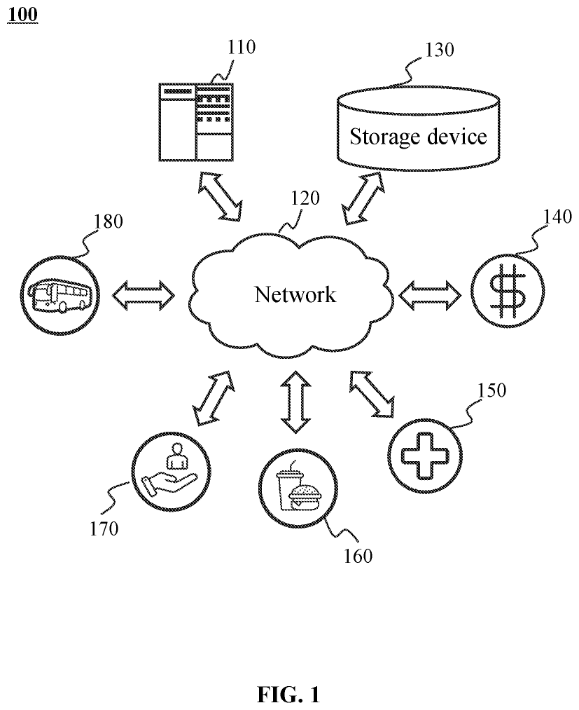

FIG. 1 is a schematic diagram of the application scenario of a system for social assistance review of a smart city based on Internet of Things according to some embodiments of the present disclosure. As shown in FIG. 1, the application scenario 100 of the system for social assistance review of the smart city based on Internet of Things may include a processing device 110, a network 120, a storage device 130, a financial management system 140, a medical system 150, a food distribution system 160, a social assistance system 170 and a bus system 180.

In some embodiments, one or more components of the application scenario 100 may be connected and/or communicate through the network 120 (such as wireless connection, wired connection, or its combination). As shown in FIG. 1, the processing device 110 may be connected to the storage device 130 through the network 120. As another example, the processing device 110 may be connected to the medical system 150 and the food distribution system 160 through the network 120

The processing device 110 may be used to process information and/or data related to the application scenario 100, such as social assistance applications, reviewing auxiliary information, etc. The processing device 110 may process the data, information, and/or processing results obtained from other devices or components of the application scenario 100, and perform program instructions based on these data, information, and/or processing results to perform one or more functions described in the present disclosure. In some embodiments, the processing device 110 may be configured to maintain and manage the social assistance management platform.

The network 120 may connect various components of the application scenario 100 and/or connect the application scenario 100 with external resources. The network 120 may allow communication between the various components and other parts outside the application scenario 100 to promote the exchange of data and/or information. The network may include a local area network (LAN), a wide area network (WAN), an internet, or the like, or any combination thereof.

The storage device 130 may be used to store data and/or instructions. In some embodiments, the storage device 130 may store data and/or instructions executed by the processing device 110 to implement an exemplary method described in the present disclosure. In some embodiments, the storage device 130 may be connected to the network 120 to communicate with one or more components of the application scenario 100 (for example, the processing device 110, and the medical system 150).

The financial management system 140 may refer to the system that provides management for finance, for example, a management system of a bank, etc. The financial management system may not only manage the funds of a user but also ensure the security of funds and personal information. For example, the financial management system may keep the funds of the user, and ensure the security of funds and personal information through encryption algorithms, biometrics, and other methods.

The financial management system may include a variety of information of a user, for example, the basic information of the user, such as a name, a mobile phone number, etc.; as another example, the deposit information of the user, such as the total deposit; as another example, the consumption information of the user, such as an amount of each consumption, an amount of consumption per month, etc.

The medical system 150 may refer to a system used in the medical system, for example, the pharmacy system, the charging system, the medical record system of the hospital. The medical system may include a variety of information of a patient, for example, the basic information of the patient, such as a name, relatives, friends, etc.; as another example, the medical record information of the patient, such as a count of times of doctor visits, a medical record, and an amount of treatment cost, or the like; as another example, the medical insurance information of the patient, such as an amount of medical insurance reimbursement, a count of medical insurance reimbursement, etc.

The food distribution system 160 may refer to a system that provides management of food distribution, for example, a system for performing to review before food distribution, a system for performing food distribution, etc. The food distribution system may include a variety of information of people who receives food, for example, the basic information of the people who receive, such as a name, relatives, friends, etc.; as another example, the receiving information of the people who receive the food, such as a count of receiving the food, a total value of the food received, a location for receiving the food, and an amount of received food.

The social assistance system 170 may refer to a system that provides management for social assistance, for example, a reviewing system, a system for implementing social assistance, or the like. The social assistance system may include a variety of information about a person who needs to be assisted and various institutions, for example, the address, income information, bank statement, credit information, fraud risk, or the like of the person who needs to be assisted. As another example, various information of a user included in the financial management system used by financial institutions, such as banks and financial management centers, various information of a patient included in the medical system used by medical insurance institutions, such as medical insurance centers.

The bus system 180 may refer to a system that provides management for buses, for example, bus scheduling system, passenger ride recording system, passenger flow statistics system, etc. The bus system may include ride recording information of a passenger, for example, a count of times of riding a bus, stations of getting on or off a bus, or the like.

It should be noted that the application scenario is only provided for illustrative purposes and is not intended to limit the scope of the present disclosure. For those skilled in the art, various modifications or changes may be made based on the description of the present disclosure. For example, the application scenario may also include a database. As another example, the application scenario may be implemented on other devices to achieve similar or different functions. However, changes and modifications do not deviate from the scope of the present disclosure.

The system for the Internet of Things is an information processing system that includes part or all of a user platform, a service platform, and a management platform. The user platform is the leader of the entire operating system of the Internet of Things, which may be configured to obtain the demand of a user. The demand of the user is a foundation and premise of the formation of the operating system of the Internet of Things. The connection between each platform of the Internet of Things is to meet the demand of the user. The service platform is a bridge between the user platform and the management platform to realize the connection of the user platform and the management platform. The service platform may provide a user with input and output services. The management platform may realize the connection and collaboration between various functional platforms (such as the user platform and the service platform). The management platform brings together information about the Internet of Things operation system, which may provide the functions of perception management and control management for the operation system of Internet of Things.

The processing of information in the Internet of Things system may be divided into the processing flow of perceptual information and the processing flow of control information. Control information may be information generated based on perceptual information. The processing of the perceptual information is that the perceptual information is passed to the service platform by the management platform and eventually reaches the user platform. The control information is issued by the user platform and reaches the management platform through the service platform.

In some embodiments, when the Internet of Things system is applied to city management, it may be called an Internet of Things system of a smart city.

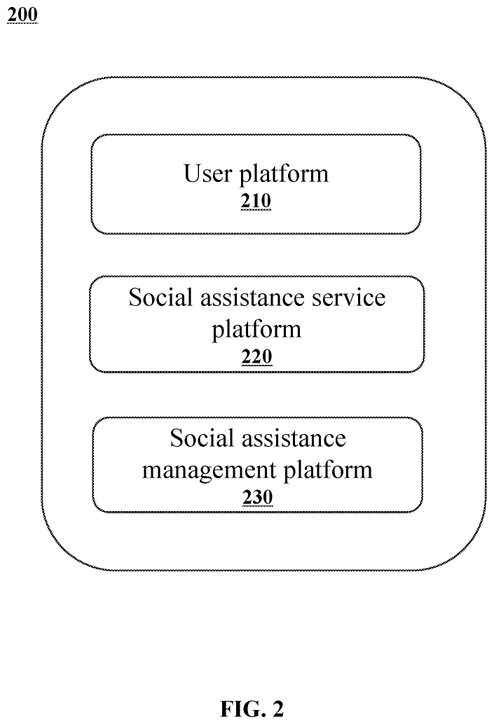

FIG. 2 is a schematic diagram illustrating an exemplary process for a system for social assistance review of a smart city based on Internet of Things according to some embodiments of the present disclosure. As shown in FIG. 2, the review system 200 for social assistance of a smart city based on Internet of Things, the following is referred to as system 200, including a user platform, a social assistance service platform, and a social assistance management platform. In some embodiments, the system 200 may be a part of the processing device 110 or implemented by the processing device 110.

In some embodiments, the system 200 may be applied to a variety of scenarios of social assistance. In some embodiments, the system 200 may respectively obtain assistance application data, assistance feedback data, and assistance implementation data in various scenarios to obtain management strategies for social assistance operations. In some embodiments, the system 200 may obtain social assistance management strategies in an entire area (such as an entire city) based on social assistance-related data obtained from each scenario.

A variety of scenarios of social assistance may include scenarios such as social assistance applications, social assistance reviewing, the distribution and management of assistance goods, etc. For example, it may include the prediction of the count of assistance applicants, the prediction of donation material, the transportation management of the assistance goods, the storage management of the assistance goods, etc. It should be noted that the above scenario is only an example, and it does not restrict the specific application scenarios of the system 200. Based on the disclosure of the embodiments, those skilled in the art may apply the system 200 to any other appropriate scenarios.

In some embodiments, the system 200 may be applied to the prediction of the count of assistance applicants. When the system 200 is applied to the prediction of the count of assistance applicants, the user platform may be configured to collect corresponding user information, such as the identity information, family information, work information, and assistance demand of candidate applicants. The social assistance service platform may summarize the information of each user to the social assistance management platform, and the social assistance management platform may analyze and process the data received. For example, the social assistance management platform may extract features of a candidate applicant according to identity information, family information, work information, assistance demand, etc., then combine with features of historical people who proposes an assistance application; predict the probability that the candidate applicant may propose an assistance application by comparing the two; and determine the candidate applicant as an applicant who will propose an assistance application when the probability value is greater than the pre-set value. Thereby a pre-evaluation of a possible count of applicants may be implemented, and further, information about the applicant such as the distribution is obtained to better carry out the reviewing work for the application. For example, the count of reviewers can be increased in areas with a large count of applicants.

In some embodiments, the system 200 may be applied to the management of the prediction of donation material. When system 200 is applied to the prediction of donation material, the user platform may be configured to collect corresponding user information, such as the current situation information of historical donors and user information of candidate donors. For example, the current situation information of historical donors and user information of candidate donors may include identity information, family information, work information, donation intentions, etc. The social assistance service platform may summarize the information of each user to the social assistance management platform, and the social assistance management platform may analyze and process the data received. For example, the social assistance management platform may determine the quasi donation user with capability and strong donation intentions firstly according to identity information, family information, work information, donation intentions, etc., and then speculate the possible donations of the quasi donation user and the corresponding prices based on historical information. At the same time, the social assistance management platform may also determine a pre-donated user with a larger possibility of donation based on analyzing the above data, and by combining feature comparison and other methods, determine the possible donations or prices of the pre-donated user.

Based on the above analysis, it may implement to obtain the types and quantities of the donation items that may be received in advance to set the corresponding acceptance scheme for different donation items. For example, for items with a short shelf life, it is necessary to deploy a distribution plan in advance, and for items that need to be refrigerated, it needs to prepare a corresponding refrigerated warehouse in advance.

In some embodiments, the system 200 may be applied to the transportation management of the assistance items When the system 200 is applied to the transportation management of the assistance items, the user platform may be configured to collect the information of donation items of donors, such as the type of donations, the weight of donations, donation addresses, etc. The social assistance service platform may summarize the information entered by each donation user to the social assistance management platform, and the social assistance management platform may analyze and process the data received. For example, the social assistance management platform may count the number of donation items that may be received in each area according to the donation address, and perform statistics on the distribution of various types of urgent materials, etc. Then based on the principle of nearby, by the optimal transportation plan, the corresponding assistance goods may be transported to the place of distribution.

In some embodiments, the system 200 may be applied to the storage management of the assistance items. When the system 200 is applied to the storage management of the assistance items, the user platform may be configured to collect the information of donation items entered by donors, such as types of items, shelf life, weight, donation address, preservation demands, etc. The social assistance service platform may summarize the information entered by each donation user to the social assistance management platform and the social assistance management platform may analyze and process the data received. For example, the social assistance management platform may determine the corresponding preservation solution according to the types of assistance items, preservation demands, amount of materials, or the like.

System 200 will be described in detail below through an example that the system 200 is applied for the review scenario of social assistance applications.

The user platform 210 may refer to a platform led by a user, including obtaining the input of the user and giving feedback on information to the user. In some embodiments, the user platform 210 may be configured to receive user demands, such as receiving a social assistance application submitted by a user.

The social assistance service platform 220 may be a platform that conveys the input of the user and control information. It connects the user platform 210 and the social assistance management platform 230.

In some embodiments, the social assistance service platform 220 may obtain the social assistance application of the assistance applicant based on the user platform 210, inquire whether the social assistance application has passed, and feedback on the result of the review to the user.

The social assistance management platform 230 may refer to a platform for the management of social assistance. In some embodiments, the social assistance management platform 230 may be configured to obtain a social assistance application of an assistance applicant based on a user platform by a social assistance service platform; obtain auxiliary reviewing information; determine an assistance object passing the review by reviewing the social assistance application based on the auxiliary reviewing information.

In some embodiments, the social assistance management platform 230 may be configured to send amount limit information of deposit and amount limit information of consumption to a financial management system; obtain a first feature of the assistance applicant based on security computing of privacy protection through the financial management system.

In some embodiments, the social assistance management platform 230 may be configured to obtain a medical knowledge graph of the assistance applicant based on a medical system; obtain a second feature of the assistance applicant based on the medical knowledge graph.

In some embodiments, the social assistance management platform 230 may be configured to send auxiliary medical information of the assistance object to the medical system.

In some embodiments, the social assistance management platform 230 may be configured to obtain a food-received knowledge graph of the assistance applicant based on a food distribution system; obtain a third feature of the assistance applicant based on the food-received knowledge graph.

In some embodiments, the social assistance management platform 230 may be configured to send auxiliary food information of the assistance object to the food distribution system.

In some embodiments, the social assistance management platform 230 may be configured to obtain an integrated information knowledge graph of the assistance applicant based on a social assistance system; obtain at least one of credit information of the assistance applicant, financial information of the assistance applicant, medical information of the assistance applicant, medical information of a related person of the assistance applicant, free food-received information of the assistance applicant, related information of a risk person of the assistance applicant, historical social assistance application information of the assistance applicant, historical social assistance application information of the related person of the assistance applicant as auxiliary risk assessment information based on the integrated information knowledge graph; obtain fraud risk information of the assistance object by performing a fraud risk assessment on the assistance object based on the auxiliary risk assessment information.

In some embodiments, the social assistance management platform 230 may be configured to determine a re-assessment assistance object based on the fraud risk information; obtain bus-riding information of the re-assessment assistance object based on a bus system; adjust the fraud risk information of the re-assessment assistance object based on the bus-riding information.

In some embodiments, the social assistance management platform 230 may be configured to adjust the fraud risk information of the re-assessment assistance object based on the bus-riding information and positioning information of the re-assessment assistance object.

In some embodiments, the social assistance management platform may be divided into multiple sub-platforms, such as a medical management sub-platform, a financial management sub-platform, a food distribution management sub-platform, a social assistance management sub-platform, and a traffic management sub-platform, or the like.

The medical management sub-platform may obtain data from a medical insurance platform. For example, the medical management sub-platform may obtain the medical insurance data of an assistance applicant from the medical insurance platform.

The financial management sub-platform may obtain data from the financial management system. For example, the financial management sub-platform may obtain the deposit and consumption data of an assistance applicant from the financial management system.

The food distribution management sub-platform may obtain data from the food distribution platform. For example, the food distribution management sub-platform may obtain data from the food distribution platform to obtain data from the assistance applicants to receive free food.

The social assistance management sub-platform may obtain data from the database of the social assistance management sub-platform. For example, the social assistance management sub-platform may obtain the past assistance information data of the assistance applicant from the database of the social assistance management sub-platform.

The traffic management sub-platform may obtain data from the database of the traffic management platform. For example, the traffic management sub-platform may obtain data of taking public transport by the assistance applicant from the database of the traffic management platform.

It should be noted that the above descriptions of the system and its components are intended to be convenient, and the present disclosure cannot be limited to the scope of the embodiments. It may be understood that for those skilled in the art, after understanding the principle of the system, it is possible to arbitrarily combine the various parts, or form a subsystem to connect with other parts without departing from the principle. For example, the social assistance service platform and the social assistance management platform may be integrated into a part. As another example, each component may share a storage device, and each component may also have its own storage device. Such deformations may be all within the scope of the protection of the present disclosure.

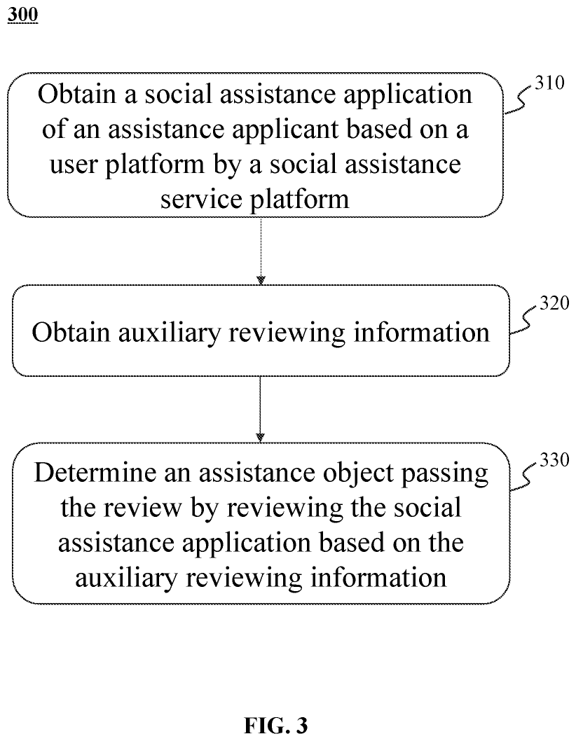

FIG. 3 is a flowchart illustrating an exemplary process for a method for social assistance review of a smart city based on Internet of Things according to some embodiments of the present disclosure. As shown in FIG. 3, process 300 includes the following steps. In some embodiments, process 300 may be performed by the social assistance management platform 230.

Step 310, obtain a social assistance application of an assistance applicant based on a user platform by a social assistance service platform.

The assistance applicant may refer to a person who applies for social assistance. For example, a low-guarantee household and a person without labor capability apply for social assistance because the basic life needs cannot be guaranteed. These people who submit the applications may be called assistance applicants.

The social assistance application may refer to application for economic and material help from the country and society, for example, the minimum living guarantee application, medical assistance application, etc.

In some embodiments, the social assistance management platform may communicate with the social assistance service platform and the user platform, and obtain a social assistance application of an assistance applicant based on the user platform by the social assistance service platform. For example, an assistance applicant may submit a social assistance application through the user platform. The social assistance service platform may obtain the social assistance application of the assistance applicant from the user platform and send it to the social assistance management platform based on the request of the social assistance management platform.

Step 320, obtain auxiliary reviewing information.

The auxiliary reviewing information may refer to information that plays an auxiliary role when a social assistance application is being reviewed, for example, income information, medical record information, etc.

In some embodiments, the social assistance management platform may obtain multiple data through multiple management sub-platforms and may use these data as the auxiliary reviewing information. For example, the social assistance management platform may obtain data from the medical system by the medical management sub-platforms, and use the data obtained as the auxiliary reviewing information. As another example, the social assistance management platform may obtain data from the financial management system through the financial management system, and use the data obtained as the auxiliary reviewing information.

In some embodiments, the social assistance management platform may obtain the auxiliary reviewing information through the following methods: sending amount limit information of deposit and amount limit information of consumption to the financial management system; obtaining a first feature of the assistance applicant based on security computing of privacy protection through the financial management system.

The amount limit information of deposit may refer to the amount information for limiting the deposit. For example, the amount limit information of deposit may include the total amount limit information of the total deposit, the amount limit information and count limit information of daily deposit and/or each deposit. For example, the amount limit information of deposit is that the total deposit is not higher than 50,000 yuan, the amount limit of each deposit is 3,000 yuan, the amount limit of the daily cumulative deposit is 5,000 yuan, and the count of the daily cumulative deposit does not exceed 5 times, etc.

In some embodiments, the social assistance management platform may determine the amount limit information of the deposit based on manual inputting. For example, if the information entered manually is that the amount limit information of deposit is that the total deposit is not higher than 50,000 yuan, the social assistance management platform may determine the total deposit being not higher than 50,000 yuan as the amount limit information of deposit.

The amount limit information of consumption may refer to information of limiting the consumption, for example, the amount limit information and count limit information of daily consumption and/or each consumption. For example, the amount limit of each consumption is 300 yuan, the amount limit of the daily cumulative consumption is 1,000 yuan, and the count of the daily cumulative consumption does not exceed 6 times, etc.

In some embodiments, the social assistance management platform may determine the amount limit information of the consumption based on manual inputting. For example, if the information entered manually is that the amount limit of each consumption is 300 yuan, the social assistance management platform may determine the amount limit of each consumption being 300 yuan as the amount limit information of consumption.

The security computing of privacy protection may refer to a computing method that may protect privacy, for example, a multi-party security computing, such as key sharing, a random prophecy machine, etc.

In some embodiments, multiple financial management systems perform security computing of privacy protection on the information included in the respective systems and obtain results. The social assistance management platform may summarize the multiple results obtained to obtain the final result, and use the final result as information of deposit and/or consumption.

Through the multi-party security computing, the information of all parties participating in the calculation may be ensured to be not disclosed under the premise of no trusted third party, accurate computing results can be obtained and the privacy of the user can be protected.

The first feature of the assistance applicant may refer to a feature indicating whether the deposit and/or consumption of the assistance applicant exceeds the limit. The first feature of the assistance applicant may have various expression methods such as words and numbers, for example, “deposit exceeds the limit”, “consumption exceeds 200% of the limit”, etc.

In some embodiments, whether the deposit and/or consumption exceeds the limit may be judged based on the amount limit information of deposit and/or consumption. In some embodiments, the financial management system may compare the deposit and/or consumption information of the user with the amount limit information of deposit and/or consumption to determine the first feature of the assistance applicant and return it to the social assistance management platform. For example, if the deposit is greater than the total deposit specified by the amount limit information of deposit, the “deposit exceeds the limit” is returned to the social assistance management platform as the first feature of the assistance applicant.

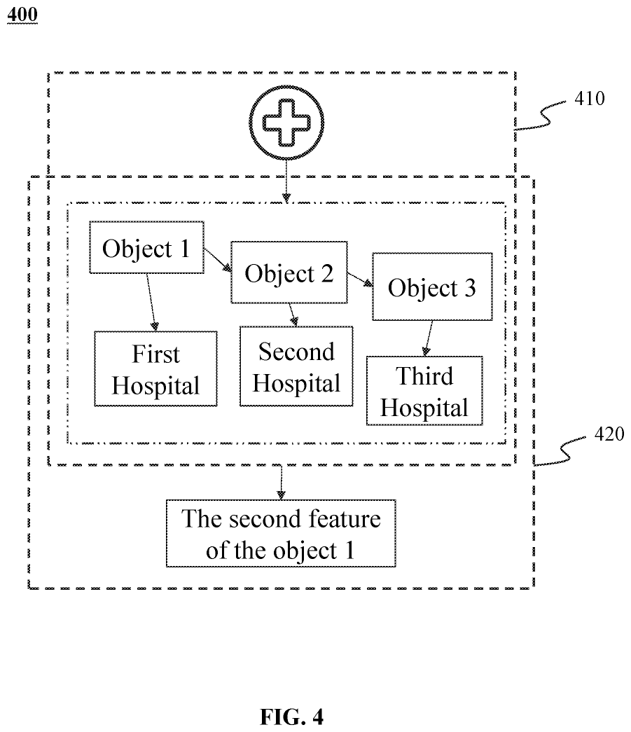

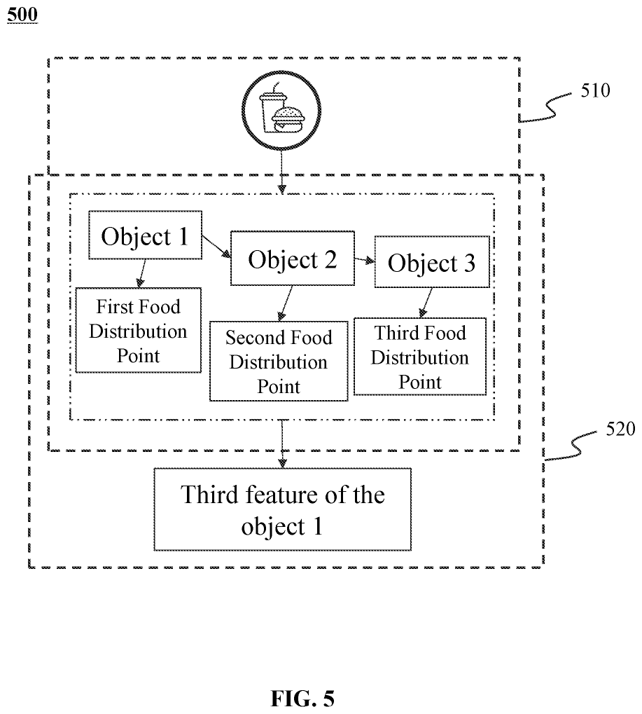

For more descriptions of obtaining auxiliary reviewing information, see FIG. 4, FIG. 5, and their descriptions.

Step 330, determine an assistance object passing the review by reviewing the social assistance application based on the auxiliary reviewing information.

The assistance object may refer to an object having received, being receiving, or being to receive social assistance, for example, a person who has passed the review of the social assistance application, a person who is receiving social assistance, etc.

In some embodiments, the social assistance management platform may determine the assistance object passing the review by reviewing the social assistance application based on the auxiliary reviewing information. In some embodiments, the social assistance management platform may determine the assistance object based on the auxiliary reviewing information that meets the preset standard. For example, the assistance applicant corresponding to the auxiliary reviewing information that meets the preset standard is determined as an assistance object that has passed the review.

The preset standard may refer to a condition preset for the auxiliary reviewing information based on experience. The preset standard may be related to the deposit information, consumption information, and medical information of the assistance applicant. For example, the preset standard may be that the first feature is that the information of deposit and consumption does not exceed the limit. If the first feature of the assistance applicant obtained above is that the information of deposit and consumption does not exceed the limit, the assistance applicant may be determined as the assistance object.

Some embodiments of the present disclosure use the auxiliary reviewing information to review the social assistance application, which can improve the accuracy of the review. At the same time, multi-party security computing can protect the privacy of the user.

FIG. 4 is a flowchart illustrating an exemplary process for a method for obtaining auxiliary reviewing information according to some embodiments of the present disclosure. As shown in FIG. 4, process 400 includes the following steps. In some embodiments, the process 400 may be performed by the social assistance management platform 230.

Step 410, obtain a medical knowledge graph of the assistance applicant based on a medical system.

The medical knowledge graph may refer to a knowledge graph related to times of doctor visits. The medical knowledge graph may include multiple nodes and multiple edges. In the medical knowledge graph, the type of nodes may include object nodes and institutional nodes. The object nodes refer to nodes that are generated based on the assistance applicant and his/her related person(s). Object nodes may also be called person nodes. The institutional nodes may refer to nodes generated based on medical institutions. For example, the medical knowledge graph corresponding to assistance applicant A may include “Object 1”, “Object 2”, “First Hospital” and other nodes, among them, “Object 1” corresponds to assistance applicant A, and “Object 2” is the father of “Object 1”.

In some embodiments, a plurality of nodes may be connected by the one or more edges, and the edges may reflect the relationship between nodes. The attributes of the edges may include the count of times of doctor visits, the count of the serious disease, the severity of the disease, the amount, the count of medical insurance reimbursement, the total amount of medical insurance reimbursement, etc. For example, “Object 1” and “First Hospital” are connected based on an edge. Based on the attributes of this edge, the information generated by “Object 1” in “First Hospital” such as the count of times of doctor visits, the count of the serious disease, the severity of the disease, the amount, the count of medical insurance reimbursement, the total amount of medical insurance reimbursement may be determined. In some embodiments, an edge of the medical knowledge graph may have a weight (not shown in the figure) to indicate the importance of the object node corresponding to a related person to the object node corresponding to the assistance applicant.

A node, the type of which is a person (i.e., the object node) in the medical knowledge graph and that has no arrows pointed to or out may be used as a root node in the medical knowledge graph. For example, as shown in FIG. 4, the node of “Object 1” may be used as the root node. This medical knowledge graph may be called the medical knowledge graph of Object 1, that is, object 1 is the assistance applicant who needs to be evaluated.

In some embodiments, the medical knowledge graph may be completed in advance and may be updated dynamically. For example, the medical knowledge graph may be established artificially in advance, and the medical knowledge graph may be updated (such as increase, delete, etc.) according to different situations (such as new diseases).

In some embodiments, the social assistance management platform may obtain the medical knowledge graph of the assistance applicant based on the identity information of the assistance applicant and the medical system. For example, based on the ID number of an assistance applicant, the social assistance management platform may find a medical knowledge graph that matches the ID number of the assistance applicant through the medical system and uses it as the medical knowledge graph of the assistance applicant.

In some embodiments, the social assistance management platform may also obtain the medical knowledge graph of the assistance applicant in other ways, for example, by marking the medical data artificially, etc.

Step 420, obtain a second feature of the assistance applicant based on the medical knowledge graph.

The second feature of the assistance applicant may refer to a feature indicating medical-related information of the assistance applicant. For example, the second feature may include the count of times of doctor visits of the assistance applicant, the count of the serious disease of the assistance applicant, the severity of the disease of the assistance applicant, the amount spent on the illness by the assistance applicant, the count of medical insurance reimbursement of the assistance applicant, the amount of medical insurance reimbursement of the assistance applicant, etc. In some embodiments, the second feature of the assistance applicant may have various expression methods such as words, numbers, etc. For example, the count of times of doctor visits of the assistance applicant is 10, the amount of cost of the assistance applicant is 123,829 yuan, and the severity of the disease of the assistance applicant is very serious.

In some embodiments, the social assistance management platform may obtain the medical-related information of the assistance applicant based on the attributes of the edges in the medical knowledge graph, and then determine the second feature of the assistance applicant.

There may be one or more object nodes in the medical knowledge graph of the assistance applicant connected with the node of the assistance applicant. These nodes may be called the relationship nodes, and a person corresponding to each of these object nodes may be called the related person. For example, as shown in FIG. 4, if “Object 1” is an assistance applicant, “Object 2” may be called the related person of “Object 1”. Due to the effects of blood relationships or other factors, the weights of the connection between multiple related persons and the assistance applicant may be different.

In some embodiments, the medical knowledge graph of the assistance applicant may also include the medical-related information of the related person, and the medical-related information of the related person may be used as a part of the medical-related information of the assistance applicant or an influencing factor thereof.

The medical-related information of the related person may refer to a variety of information related to the treatment of the disease of the related person, for example, the count of times of doctor visits, the count of the serious disease, the severity of the disease, the amount of cost, the count of medical insurance reimbursement, the amount of medical insurance reimbursement, etc.

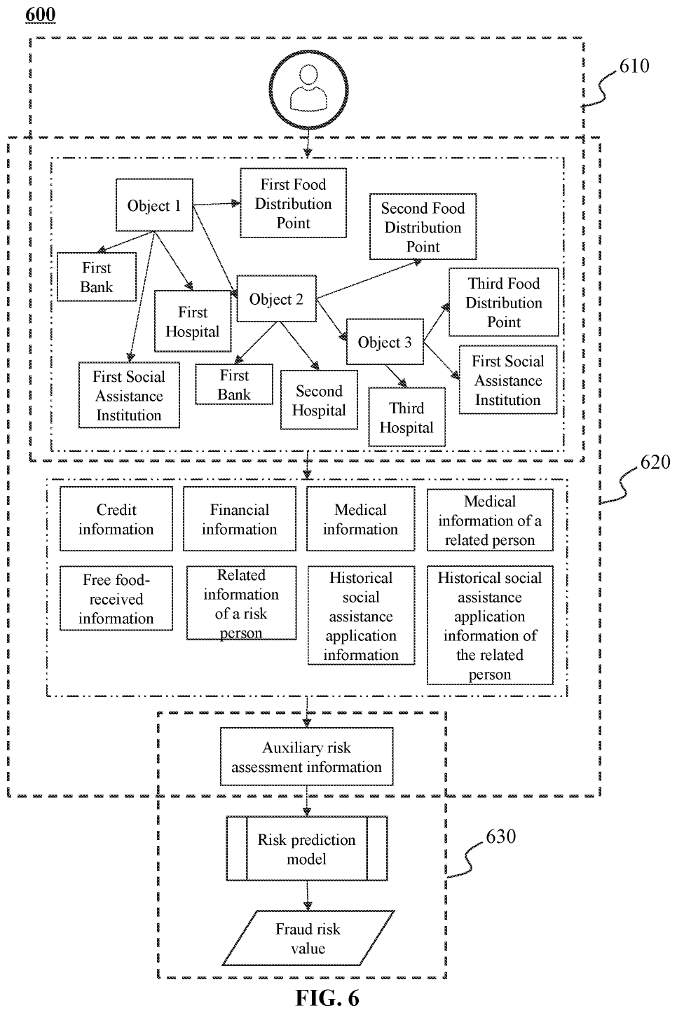

In some embodiments, the social assistance management platform may, starting from the corresponding node of the assistance applicant in the medical knowledge graph, obtain object nodes connected to edges of adjacency degree being larger than or equal to 1, find the attributes of the edges connecting these nodes to the node of the medical institution, and obtain the medical-related information of the related person. The adjacency degree may be used to indicate the degree of the adjacency between nodes or between edges and nodes. The edge that the adjacent degree is 1 refers to the edge starting from the current node. For more descriptions of the adjacency degree, see FIG. 6 and its descriptions.

In some embodiments, the social assistance management platform may summarize the medical-related information of the assistance applicant and the medical-related information of the related person to obtain the second feature of the assistance applicant. For example, if the second feature contains the count of times of doctor visits, that the count of times of doctor visits of object 1 is 1 is determined based on the attribute of the edge connected to the medical institution node of the edges of the “object 1” that the adjacency degree is 1, and that the count of times of doctor visits of object 2 is 20 is determined based on the attribute of the edge connected to the medical institution node of the edges of the “object 2” that the adjacency degree is 1, then the social assistance management platform may add the count of times of doctor visits of object 1 and the count of times of doctor visits of object 2 to obtain the sum result 21, and use the result 21 as the information of the count of times of doctor visits in the second feature of the assistance applicant, that is, the count of times of doctor visits in the second feature of the assistance applicant (e.g., object 1) is 21. The obtaining methods of other information in the second feature such as the count of the serious disease and the medical amount are the same as that of the count of times of doctor visits, which are omitted here.

In some embodiments, the social assistance management platform may weight the medical-related information of one or more related persons, and then add the weighted result with the medical-related information of the assistance applicant to obtain the second feature of the assistance applicant. Still taking the above example, if the medical knowledge graph of the assistance applicant also includes “Object 3”, “Object 3” and “Object 2” are father and daughter, and “Object 3” and “Object 1” are connected by the edge that the adjacency degree is 2 (that is, “Object 3” and “Object 1” are separated by “Object 2”), that the count of times of doctor visits of object 3 is 30 is determined based on the attribute of the edge (that is, the edge connecting “Object 3” and “Third Hospital” in FIG. 4) connected to the medical institution node connected to the edges of the “object 3” that the adjacency degree is 1.

In some embodiments, the corresponding weight of the corresponding medical-related information of each related person is related to the adjacency degree between its edge and the root node. For example, the smaller the adjacency degree, the closer the relationship, and the larger the corresponding weight. For example, if the weight between “Object 1” and “Object 3” is 0.1, the weight between “Object 1” and “Object 2” is 0.8, the social assistance management platform may weight the count of times of doctor visits of object 2 and the count of times of doctor visits of object 3 to obtain the weighted result 19, the count of times of doctor visits of object 1 is added to the weighted result to obtain the sum result 20, and the result 20 is used as the count of times of doctor visits in the second feature of the assistance applicant, that is, the count of times of doctor visits in the second feature of the object 1 is 20. The obtaining methods of other information in the second feature such as the count of the serious disease and the medical amount are the same as that of the count of times of doctor visits, which are omitted here.

In process 300, when reviewing the social assistance application based on the auxiliary reviewing information, the preset standard described in step 330 may be related to the second feature. For example, the preset standard includes that the count of times of doctor visits is larger than N, and the count of the serious disease is larger than M. It may be understood that if the count of times of doctor visits of the assistance applicant is larger than N, and the count of the serious disease of the assistance applicant is larger than M, it may be considered that the physical condition of the assistance applicant is poor and the assistance needs to be provided for the assistance applicant.

In some embodiments, the process 400 may also include sending auxiliary medical information of the assistance object to the medical system. It should be noted that this step is a non-necessary step, which may be set according to actual needs.

The auxiliary medical information may refer to information related to times of doctor visits received by the assistance object, for example, name, age, ID number, past medical history, etc.

In some embodiments, the social assistance management platform may send auxiliary medical information of the assistance object to the medical system. This setting can make the medical system not need to review the social assistance application submitted by the person who has passed the social assistance application again, so that the person can enjoy medical assistance in time.

Some embodiments of the present disclosure judge the degree that the assistance applicant needs to be assisted based on the condition of the physical health of the assistance applicant and his related person, for example, a person who is healthy usually does not apply for social assistance, which can further improve the accuracy of the review of the assistance applicant.

It should be noted that the description of the above-mentioned process 400 is only for examples and descriptions, but does not limit the scope of the application of the present disclosure. For those skilled in the art, various modifications and changes may be made to the process 400 under the guidance of the present disclosure. However, these modifications and changes are still within the scope of the present disclosure.

FIG. 5 is a flowchart illustrating an exemplary process for obtaining auxiliary reviewing information according to the other embodiments of the present disclosure. As shown in FIG. 5, the process 500 includes the following steps. In some embodiments, the process 500 may be performed by the social assistance management platform 230.

Step 510, obtain a food-received knowledge graph of the assistance applicant based on a food distribution system.

The food-received knowledge graph may refer to a knowledge graph related to receiving the food. The food-received knowledge graph may include multiple nodes and multiple edges. Similar to the medical knowledge graph, in the food-received knowledge graph, the type of nodes may include person nodes (that is, object nodes) and institutional nodes. The institutional nodes may refer to nodes generated based on food distribution institutions. For example, the food-received knowledge graph corresponding to the assistance applicant A may include “Object 1”, “Object 2” and other object nodes, as well as “First Food Distribution Point” and “Second Food Distribution Point” and other institutional nodes. “Object 1” corresponds to assistance applicant A, “Object 2” is the father of “Object 1”.

In some embodiments, a plurality of nodes may be connected by the edges, and the edges may reflect the relationship between nodes. The attributes of the edges may include the count of times of receiving the food, the total amount of the food received, the place to receive the food, and the count of the food received. For example, “Object 1” and “First Food Distribution Point” are connected based on an edge. Based on the attribute of this edge, the information generated by “Object 1” in “First Food Distribution Point,” such as the count of times of receiving the food (e.g., free food), the total amount of the food received, the place to receive the food, and the count of the food received may be determined. In some embodiments, the edges of the food-received knowledge graph may have weights (not shown in the figure) to indicate the importance of the object node corresponding to a related person to the object node corresponding to the assistance applicant.

A node, the type of which is a person (i.e., the object node) in the food-received knowledge graph, and which has no arrow pointed to or out may be used as the root node in the food-received knowledge graph. For example, as shown in FIG. 5, the node of “Object 1” may be used as the root node. This food-received knowledge graph may be called the food-received knowledge graph of Object 1, that is, object 1 is the assistance applicant who needs to be evaluated.

In some embodiments, the food-received knowledge graph may be completed in advance and may be updated dynamically. For example, the food-received knowledge graph may be established artificially in advance, and the food-received knowledge graph may be updated (such as increase, delete, etc.) according to different situations (such as receiving new foods).

In some embodiments, the social assistance management platform may obtain identity information of a receiving person based on information such as an identification code binding with identity identification and discount information used by the receiving person when receiving food. Information such as an identification code binding with identity identification and discount information may refer to information binding with identity. Through this information, the only identity information of the receiving person may be determined. For example, the ID number may be determined through the identification code.

In some embodiments, the social assistance management platform may obtain the food-received knowledge graph of the assistance applicant based on the identity information of the assistance applicant and the food distribution system. For example, based on the ID number of the assistance applicant, the social assistance management platform may find a food-received knowledge graph that matches the ID number through the food distribution system and uses it as the food-received knowledge graph of the assistance applicant.

In some embodiments, the social assistance management platform may also obtain the food-received knowledge graph of the assistance applicant through other methods, for example, marking the receiving data by manually, etc.

Step 520, obtain a third feature of the assistance applicant based on the food-received knowledge graph.

The third feature of the assistance applicant may refer to the feature indicating information related to receiving the food of the assistance applicant. For example, the third feature may include the count of times of receiving the food by the assistance applicant, the total amount of the food received by the assistance applicant, the place to receive the food by the assistance applicant, the count of food received by the assistance applicant, etc. In some embodiments, the third feature of the assistance applicant may have various expression methods such as words and numbers. For example, the count of times of receiving the food by the assistance applicant is 6, the total amount of food received by the assistance applicant is 885 yuan, etc.

In some embodiments, the social assistance management platform may obtain the information related to receiving food of the assistance applicant based on the attributes of the edges in the food-received knowledge graph.

There may be one or more object nodes in the food-received knowledge graph of the assistance applicant, which are connected with the node of the assistance applicant. These nodes may be called the relationship nodes, and the persons corresponding to these object nodes may be called the related persons. For example, as shown in FIG. 5, if “Object 1” is an assistance applicant, “Object 2” may be called the related person of “Object 1”. Due to the effects of blood relationships or other factors, the weights of the connection between multiple related persons and the assistance applicant may be different.

In some embodiments, the food-received knowledge graph of the assistance applicant may also include the information related to receiving food of the related person, and the information related to receiving food of the related person may be used as a part of the information related to receiving food of the assistance applicant or an influencing factor thereof.

The information related to receiving food of the related person may refer to a variety of information related to receiving food by the related person, for example, the count of times of receiving the food, the total amount of the food received, the place to receive the food, the count of receiving the food, etc.

In some embodiments, the social assistance management platform may obtain object nodes connected to edges that adjacency degrees are larger than or equal to 1 by starting from the node corresponding to the assistance applicant in the food-received knowledge graph, find the attributes of the edges connecting these nodes with the node of the food distribution institution, and obtain the information related to receiving food of the related person.

In some embodiments, the social assistance management platform may summarize the information related to receiving food of the assistance applicant and the information related to receiving food of the related person to obtain the third feature of the assistance applicant. For example, if the third feature contains the count of times of receiving the food, that the count of receiving the food by object 1 is 6 is determined based on the attribute of the edge (as shown in FIG. 5, the edge connecting “Object 1” to “First Food Distribution Point”) connected to the food distribution institution node connected to the edge of the “Object 1” that the adjacency degree is 1, that the count of times of receiving the food by object 2 is 10 is determined based on the attribute of the edge connected to the food distribution institution node in the edges of the “Object 2” that the adjacency degree is 1. Then, the social assistance management platform may add the count of times of receiving the food by object 1 with the count of times of receiving the food by object 2 to obtain the sum result of 16, and use the result 16 as the information of count of times of receiving the food in the third feature of the assistance applicant, that is, the count of times of receiving the food in the third feature of the object 1 is 16. The obtaining methods of other information in the third feature such as the total amount of the food received, the place to receive the food, and the count of receiving the food are the same as that of the count of times of receiving the food, which are omitted here.

In some embodiments, the social assistance management platform may weight the information related to receiving food of one or more related persons, and then add the weighted result with the information related to receiving food of the assistance applicant to obtain the third feature of the assistance applicant. Still taking the above example, if the food-received knowledge graph of the assistance applicant also includes “Object 3”, “Object 3” and “Object 2” are father and daughter, “Object 3” and “Object 1” are connected by the edge that the adjacency degree is 2 (that is, “Object 3” and “Object 1” are separated by “Object 2”), that the count of times of receiving the food of object 3 is 30 is determined based on the attribute of the edge connected to the food distribution institution node which is connected to the edge of the “Object 3” that the adjacency degree is 1.

In some embodiments, the corresponding weight of the corresponding information related to receiving food of each related person is related to the adjacency degree between its edge and the root node. For example, the smaller the adjacency degree, the closer the relationship, and the larger the corresponding weight. For example, the weight between “Object 1” and “Object 2” is 0.9, the weight between “Object 1” and “Object 3” is 0.2, and the social assistance management platform may weight the count of times of receiving the food by object 2 and the count of times of receiving the food by object 3 to obtain the weighted result of 11, and then add the count of times of receiving the food by object 1 to the weighted result to obtain the sum result of 17, which is used as the count of times of receiving the food in the third feature of the assistance applicant, that is, the count of times of receiving the food in the third feature of object 1 is 17. The obtaining methods of other information in the third feature such as the total amount of the food received, the place to receive the food, and the count of the received food are the same as that of the count of times of receiving the food, which are omitted here.

In process 300, when reviewing the social assistance application based on the auxiliary reviewing information, the preset standard described in the step 330 may be related to the third feature. For example, the preset standard includes that the count of times of receiving the food is smaller than K, and the count of food received is smaller than P. It may be understood that if the count of times of receiving the food is smaller than K, and the count of received food is smaller than P, it may be considered that the food source of the assistance applicant mainly depends on the food received and the assistance applicant needs to be assisted.

In some embodiments, the process 500 may also include sending auxiliary food information of the assistance object to the food distribution system. It should be noted that this step is a non-necessary step, which may be set according to actual needs.

The auxiliary food information may refer to information related to the assistance object receiving food assistance, for example, name, ID number, food required, etc.

In some embodiments, the social assistance management platform may send auxiliary food information of the assistance object to the food distribution system, which can make the food distribution system not need to review the food assistance to the person who has passed the social assistance application again, so that the person can receive food assistance in time.

Some embodiments of the present disclosure judge the degree of the assistance applicant and his related person to need the assistance based on the condition of receiving food by the assistance applicant and his/her related person, for example, a person who receives free food everywhere usually has a higher risk of fraud, which can further improve the accuracy of the review of the assistance applicant.

It should be noted that the description of the above-mentioned process 500 is only for examples and descriptions, but does not limit the scope of the application of the present disclosure. For those skilled in the art, various modifications and changes may be made to the process 500 under the guidance of the present disclosure. However, these modifications and changes are still within the scope of the present disclosure.

It may be understood that the time required to review is short, and it may take a few minutes, hours, or days to complete. In order to prevent the occurrence of cheating for assistance, various information of the assistance applicant may be tracked and collected for a long time, analyzed and processed to provide social assistance to those who really need it.

FIG. 6 is a flowchart illustrating an exemplary process for obtaining fraud risk information according to some embodiments of the present disclosure. As shown in FIG. 6, process 600 includes the following steps. In some embodiments, the process 600 may be performed by the social assistance management platform 230.

Step 610, obtain an integrated information knowledge graph of the assistance applicant based on a social assistance system.

The integrated information knowledge graph may refer to a knowledge graph including part or all information in the financial management system, the medical knowledge graph, and the food-received knowledge graph. The integrated information knowledge graph is similar to the medical knowledge graph and food-received knowledge graph described earlier, which may include multiple nodes and multiple edges. In the integrated information knowledge graph, the type of nodes may include object nodes and institutional nodes. The object nodes refer to nodes that are generated based on the assistance applicant and his/her related person. Object nodes may also be called person nodes. The institutional nodes may refer to nodes generated based on financial institutions, medical institutions, and food distribution institutions. For example, the integrated information knowledge graph corresponding to the assistance applicant A may include object nodes such as “Object 1”, “Object 2”, and institution nodes such as “First Bank”, “First Hospital”, and “First Food Distribution Point”, among them, “Object 1” corresponds to assistance applicant A, and “Object 2” is the father of “Object 1”.

In some embodiments, a plurality of nodes may be connected by the edges, and the edges may reflect the relationship between nodes. The attributes of the edges may include the deposit information, the consumption information, the count of times of times of doctor visits, the count of the serious disease, the severity, the amount, the count of medical insurance reimbursement, the total amount of medical insurance reimbursement, the count of times of receiving the food, the total amount of the food received, the place to receive the food, and the count of receiving the food, etc. For example, the “Object 2” and “Second Food Distribution Point” are connected based on an edge. Based on the attributes of this edge, the information generated by “Object 2” in “Second Food Distribution Point,” such as the count of times of receiving the food, the total amount of the food received, the place to receive the food, and the count of receiving the food may be determined. In some embodiments, an edge of the integrated information knowledge graph may have a weight (not shown in the figure) to indicate the importance of the object node corresponding to a related person to the object node corresponding to the assistance applicant.

A node (i.e., the object node), the type of which is a person in the integrated information knowledge graph, and which has no arrows pointed to/out may be used as the root node in the integrated information knowledge graph. For example, as shown in FIG. 6, the node of “Object 1” may be used as the root node, and this corresponding integrated information knowledge graph may be called the integrated information knowledge graph of Object 1, that is, object 1 is the assistance applicant who needs to be evaluated. In some embodiments, the integrated information knowledge graph may be completed in advance and may be updated dynamically.

In some embodiments, nodes may also have attributes. The attributes of the nodes may refer to a general term indicating the properties of the nodes. In some embodiments, the attributes of person nodes may include address information, bank statements, income information, credit reporting data, and fraud risk value, etc. The fraud risk value may refer to the possibility of performing fraud on the assistance application. The fraud risk value may be represented by a number between 0 and 1. The larger the number, the more likely to perform fraud. For further explanation of the fraud risk value and the acquisition manner thereof, see step 630. The attributes of institutional nodes (or referred to as institution nodes) may include information such as addresses of the institutions and areas the institutions belonged to.

In some embodiments, the attributes of person nodes and institutional nodes may be obtained by reading related information stored in the storage device. For example, the storage device stores Object 1's address information, bank statements, income information, credit reporting data, fraud risk value, etc. The attribute of the “Object 1” may be obtained by reading the above information in the storage device.

In some embodiments, the attributes of person nodes and the attributes of institutional nodes may also be obtained in other ways, for example, through the Internet, manual labeling, etc.

In the integrated information knowledge graph, the type of edges may be divided into at least six types: “direct relatives”, “same address”, “statements”, “medical-related”, “receiving free food”, and “assistance application”.

Edges may also have attributes. The attributes of the edges may refer to the general term representing the relationship between nodes, for example, the subordinate relationship of the two nodes, etc. In some embodiments, the attributes of the edges may represent their own feature data. For example, as shown in FIG. 6, the attributes of the edge connecting “Object 1” and “First Bank” may represent the deposit information and withdrawal information of object 1 in the first bank.

The edges of the “direct relatives” type may be the edges of connecting person nodes and person nodes. The edges of the “direct relatives” type may indicate the relative relationships between persons. The attributes of the edges of the “direct relatives” type may include father and son, mother and son, husband and wife, etc. For example, the edge connecting “Object 1” and “Object 2” in FIG. 6 belongs to the edges of the “direct relatives” type, and the attribute of the edge in FIG. 6 are father and son.

The edges of the “same address” type may be the edges of connecting person nodes and person nodes. The attributes of the edges of the “same address” type may indicate that the address of the persons is the same. For example, by querying the address information of person A and person B, if finding that they live in the same house, node A and node B have an edge of the “same address” type. For example, the edge connecting “Object 2” and “Object 3” in FIG. 6 belongs to the edges of the “same address” type, and the attributes of the edge in FIG. 6 are the same address.

The edges of the “statement” type may be the edges connecting person nodes and the financial institution nodes. The edges of the “statement” type may indicate the statements of person and financial institutions. Based on the attributes of edges of this type, the feature of deposit and the feature of consumption of object nodes may be determined, such as deposit amount and average monthly consumption, etc. The computing method may adopt security computing of privacy protection described in FIG. 3. The attributes of edges of the “statement” type may include the occurrence time, the amount of the statement, and the content of the statement (such as, withdrawal, deposit, consumption), etc. For example, the edge connecting “Object 1” and “First Bank” in FIG. 6 belongs to the edges of the “statement” type, and the attributes of the edge in FIG. 6 include the occurrence time, the amount of the statement, and the content of the statement.

The edges of the “medical-related” type may be the edges connecting person nodes and medical institution nodes. The edges of the “medical-related” type may indicate the medical-related information of persons. The attributes of the edges of the “medical-related” type may include the count of times of doctor visits, the count of times of the serious disease, the severity of the disease, the amount of the cost, the count of medical insurance reimbursement, and the total amount of medical insurance reimbursement, etc. For example, the edge connecting “Object 1” and “First Hospital” in FIG. 6 belongs to the edges of the “medical-related” type, and the attributes of the edge in FIG. 6 include the count of times of doctor visits, the count of the serious disease, the severity of the disease, the amount of the cost, the count of medical insurance reimbursement, and the total amount of medical insurance reimbursement, etc.

The edges of the “receiving free food” type may be edges connecting person nodes and free food distribution institution nodes. The edges of the “receiving free food” type may indicate the information related to receiving free food by persons. For example, user A has been gone to the free food distribution institution B to receive free food, and to the free food distribution institution C to receive free food. There is an edge of the “receiving free food” type both between node A and node B, and between node A and node C. The attributes of the edges of the “receiving free food” type may include the receiving times (or frequency), the total amount of the food received, the place to receive, the amount of receiving the food, etc. For example, the edge connecting “Object 1” and “First Food Distribution Point” in FIG. 6 belongs to the edges of the “receiving free food” type, and the attributes of the edge in FIG. 6 include the receiving times (or frequency), the total amount of the food received, the place to receive the food, the amount of receiving the food, etc.

The edges of the “assistance application” type may be the edges connecting person nodes and the social assistance institution nodes. The edges of the “assistance application” type may indicate the event of the assistance application of the person. For example, user A has submitted an assistance application to assistance institution B and assistance institution C. There is an edge of the “assistance application” type both between node A and node B, and between node A and node C. The attributes of the edges of the “assistance application” type may include the frequency (or the times) of the assistance application, the total amount of the assistance application, the approval rate of the assistance application (or the times), etc. For example, the edge connecting “Object 1” and “First Social Assistance Institution” in FIG. 6 belongs to the edges of the “assistance application” type, and the attributes of the edge in FIG. 6 include the frequency (or the times) of the assistance application, the total amount of the assistance application, the approval rate of the assistance application (or the times), etc.

It should be noted that the above-mentioned medical knowledge graph, food-received knowledge graph, and integrated information knowledge graph may be stored in any position. For example, all are stored in the storage device 130. As another example, they are stored in one or more systems of the financial management system 140, the medical system 150, the food distribution system 160, the social assistance system 170, and the bus system 180. The same knowledge graph may be used in some cases. For example, the integrated information knowledge graph may be used in anywhere the knowledge graph is needed. Only needing to selecting the type of edges or the type of nodes, the required information may be obtained. For example, when the integrated information knowledge graph is used to obtain the second feature corresponding to medical-related information, person nodes and medical institution nodes need to be selected, or the edges belonging to “medical-related” and nodes connected by the edges need to be selected.

In some embodiments, the social assistance management platform may obtain the integrated information knowledge graph of the assistance applicant based on the identity information of the assistance applicant and the social assistance system. For example, based on the ID number of the assistance applicant, the social assistance management platform may find an integrated information knowledge graph that matches the ID number through the social assistance system and uses it as the integrated information knowledge graph of the assistance applicant.

In some embodiments, the social assistance management platform may also obtain the integrated information knowledge graph of the assistance applicant through other methods, for example, marking a variety of data manually.

Step 620, obtain at least one of credit information of the assistance applicant, financial information of the assistance applicant, personal medical information of the assistance applicant, medical information of the related person of the assistance applicant, free food received information of the assistance applicant, related information of a risk person of the assistance applicant, personal historical social assistance application information of the assistance applicant, historical social assistance application information of the related person of the assistance applicant as auxiliary risk assessment information based on the integrated information knowledge graph.

Credit information may refer to information that reflects the credit of a person, for example, credit reporting information, administrative punishment information, and court-forced enforcement information.

Financial information may refer to a variety of information related to financial activities, for example, consumption information, deposit information, financial securities transaction information, etc.

Personal medical information may refer to information related to medical-related information of a person, for example, the count of getting sick, the hospital of times of doctor visits, the count of times of medical insurance reimbursement, etc.

The medical information of the related person may refer to the medical-related information of the person related to the assistance applicant (that is, the related person), for example, the count of getting sick of the assistance applicant's son, the hospital of the times of doctor visits of the assistance applicant's father, the count of times of the medical insurance reimbursement of the assistance applicant's father, etc.

Free food-received information may refer to information related to receiving free food by the assistance applicant and/or the related person. For example, the count of times of receiving free food, the total amount of the free food received, the place to receive free food, etc.

The related information of the risk person may refer to the relationship between the assistance applicant and the person with a fraudulent risk. For example, if the father of the assistance applicant is judged by the court as a dishonest person, the related information of the risk person may be that the father of the assistance applicant is a dishonest person.

In some embodiments, the social assistance management platform may compare the fraud risk value with the preset threshold to determine the person with a fraud risk. For example, if the fraud risk value in the attributes of node “Object 2” in FIG. 6 is 0.8, the social assistance management platform compares it with the preset threshold of 0.6. If the fraud risk value exceeds the preset threshold, the social assistance management platform may determine object 2 as the person with a fraud risk. Further, the social assistance management platform may determine that object 1 is associated with the risk person object 2, and the relationship is the father and son.

In some embodiments, the assistance management platform may also determine the person with a fraud risk in other ways, for example, marking manually, etc.

Personal historical social assistance application information may refer to information related to the social assistance application submitted by the assistance applicant in a past period, for example, the count of times of the social assistance application, the time of the social assistance application, etc.

Historical social assistance application information of the related person may refer to the information related to the social assistance application submitted by the person related to the assistance applicant (that is, the related person) in a past period, for example, the count of times of the social assistance application of the assistance applicant's father, the time of the social assistance application of the assistance applicant's son, etc.

The auxiliary risk assessment information may refer to information used to assist risk assessment. For example, one or more information of credit information, financial information, personal medical information, medical information of the related person, free food-received information, related information of the risk person, personal historical social assistance application information, historical social assistance application information of the related person may be determined as auxiliary risk assessment information.

In some embodiments, the social assistance management platform may directly query the corresponding nodes and edges from the integrated information knowledge graph to obtain the auxiliary risk assessment information of the assistance applicant.

In some embodiments, the social assistance management platform may directly query the attributes of the node of the assistance applicant from the integrated information knowledge graph to obtain the credit information of the assistance applicant. For example, the social assistance management platform may find the attributes of the node of the assistance applicant (such as the “Object 1” node in FIG. 6), and determine the attributes of the node, such as bank statement, income information, and credit reporting data, as the credit information of the assistance applicant.

In some embodiments, the social assistance management platform may inquire about the attributes of the edge that the adjacency degree is 1 and the type is the “statement” type of the node corresponding to the assistance applicant from the integrated information knowledge graph to obtain the financial information of the assistance applicant. For example, the social assistance management platform may find the node corresponding to the assistance applicant (such as the “Object 1” node in FIG. 6) and determine the attributes of the edge that the adjacency degree is 1 and the type is the “statement” type (such as the edge connecting the “Object 1” node and the “First Bank” node in FIG. 6) as the financial information of the assistance applicant, such as the occurrence time, the amount of the statement, etc.

The adjacency degree may represent the degree of nodes adjacent to the edges. The unit of the adjacency degree may be a non-zero natural number. The edge that the adjacency degree is 1 may refer to the edge starting from the node (e.g., the node of the assistance applicant). For example, as shown in FIG. 6, the “First Food Distribution Point” and “Object 1” are connected through the edge that the adjacency degree is 1, and “Second Food Distribution Point” and “Object 1” are connected through the edge that the adjacency degree is 2. The larger the number of the adjacency degree, the weaker the relationship with the node.

In some embodiments, when there are multiple edges that the adjacency degree with the node of the assistance applicant is 1 and the type is the “statement” type in the integrated information knowledge graph, the social assistance management platform may use methods such as performing a sum and weighted average calculation to obtain the financial information of the assistance applicant.

In some embodiments, the social assistance management platform may inquire about the attributes of the edge that the adjacency degree is 1 and the type is the “medical-related” type of the node corresponding to the assistance applicant from the integrated information knowledge graph to obtain the personal medical information of the assistance applicant. For example, the social assistance management platform may find the node corresponding to the assistance applicant (such as the “Object 1” node in FIG. 6) and determine the attributes (such as the count of times of doctor visits, the count of times of the serious disease, etc.) of the edge that the adjacency degree is 1 and the type is the “medical-related” type (such as the edge connecting the “Object 1” node and the “First Hospital” node in FIG. 6) as the personal medical information of the assistance applicant. As shown in FIG. 6, the personal medical information of object 1 may be obtained based on the attributes of the edge between “Object 1” and “First Hospital”, which includes that the count of doctor visits is 10, the count of times of the serious disease is 3, and the severity of the disease is very serious, the amount is 50,000 yuan, the count of medical insurance reimbursement is 9, and the total amount of medical insurance reimbursement is 30,000 yuan, etc.

In some embodiments, when there are multiple edges that the adjacency degree with the node of the assistance applicant is 1 and the type is the “medical-related” type in the integrated information knowledge graph, the social assistance management platform may use methods such as performing a sum and weighted average calculation to obtain the personal medical information of the assistance applicant.

In some embodiments, the social assistance management platform may, starting from the node corresponding to the assistance applicant in the integrated information knowledge graph, obtain object nodes connected to the edges that the adjacency degree is larger than or equal to 1, and find the attributes of the edges which are connected to these object nodes and the type of which is the “medical-related” type to obtain the medical information of the related person of the assistance applicant.

For example, as shown in FIG. 6, the social assistance management platform may obtain an object node (that is, the node of “Object 2”) connected to the edge that the adjacency degree is 1 based on the node of the assistance applicant (such as, “Object1”), and find the attributes of the edge which is directly connected to “Object 2” and the type of which is “medical-related” (that is, the edge that the adjacency degree with “Object 2” is 1 and the type is “medical-related”). The attributes of the edge, such as the count of times of medical insurance reimbursement and the amount of medical insurance reimbursement, are determined to be the medical information of the related person of the assistance applicant. As shown in FIG. 6, the medical information of the related person of the assistance applicant may be obtained based on the attributes of the edge between “Object 2” and “Second Hospital”, which includes that the count of times of doctor visits is 3, the count of the serious disease is 1, and the severity of the disease is not serious, the amount is 20,000 yuan, the count of medical insurance reimbursement is 1, and the total amount of medical insurance reimbursement is 10,000 yuan, etc.

In some embodiments, when there are object nodes connected to the node of the assistance applicant by multiple edges that the adjacency degree is larger than or equal to 1 in the integrated information knowledge graph, and each object node has an edge connected directly to the node of the assistance applicant and the type is the “medical-related”, the social assistance management platform may use methods such as performing a sum and weighted average calculation on the features related to medical information in the attribute information corresponding to the multiple edges to obtain the medical information of the related person of the assistance applicant.

In some embodiments, the social assistance management platform may inquire about the attributes of the edge, that the adjacency degree is 1 and the type is the “receiving free food” type, of the node corresponding to the assistance applicant from the integrated information knowledge graph to obtain the free food-received information of the assistance applicant. For example, the social assistance management platform may find the node corresponding to the assistance applicant (such as the “Object 1” node in FIG. 6) and determine the attributes of the edge that the adjacency degree is 1 and the type is the “receiving free food” type (such as the edge connecting the “Object 1” node and the “First Food Distribution Point” node in FIG. 6) as the free food-received information of the assistance applicant, such as the count of times of receiving free food, the total amount of the free food received, etc. As shown in FIG. 6, the free food-received information of object 1 may be obtained based on the attributes of the edge between “Object 1” and “First Food Distribution Point”, which includes that the count of times of receiving free food is 16, the total value of the food received is 500 yuan, the place to receive the food is Xiang he Street, and the count of receiving free food is 22, etc.

In some embodiments, when there are multiple edges that the adjacency degree with the node of the assistance applicant is 1 and the type is the “receiving free food” type in the integrated information knowledge graph, the social assistance management platform may use methods such as performing a sum and weighted average calculation on the features related to the free food-received information in the attribute information corresponding to multiple edges to obtain the free food-received information of the assistance applicant.

In some embodiments, the social assistance management platform may, starting from the node corresponding to the assistance applicant in the integrated information knowledge graph, obtain object nodes connected to the edges that the adjacency degree is larger than or equal to 1, and find the attributes of the edges which are connected to these object nodes, and the type of which is the “receiving free food” type to obtain the free food-received information of the related person of the assistance applicant.

For example, as shown in FIG. 6, the social assistance management platform may obtain an object node (that is, the node of “Object 2”) connected to the edge that the adjacency degree is 1 based on the node of the assistance applicant (such as, node of “Object1”), and find the attributes of the edge that is directly connected to “Object 2” and the type of that is “receiving free food” (that is, the edge that the adjacency degree with “Object 2” is 1 and the type is “receiving free food”). And the attributes of the edge, such as the count of times of receiving free food, and the total value of the free food received, are determined as the free food-received information of the related person of the assistance applicant. As shown in FIG. 6, the free food-received information of the related person of the assistance applicant may be obtained based on the attributes of the edge between “Object 2” and “Second Food Distribution Point”, which includes that the count of times of receiving free food is 5, the total value of the free food received is 200 yuan, etc.

In some embodiments, when there are object nodes connected to the node of the assistance applicant by multiple edges that the adjacency degree is larger than or equal to 1 in the integrated information knowledge graph, and each object node has an edge connected directly thereto and the type is the “receiving free food”, the social assistance management platform may use methods such as performing a sum and weighted average calculation on the features related to free food-received information in the attribute information corresponding to the multiple edges to obtain the free food-received information of the related person of the assistance applicant.

In some embodiments, the social assistance management platform may, starting from the node corresponding to the assistance applicant in the integrated information knowledge graph, obtain object nodes connected to the edges that the adjacency degree is larger than or equal to 1, and perform weighted sum of the fraud risk values of these object nodes to obtain related information of risk person of the assistance applicant. For example, as shown in FIG. 6, the social assistance management platform may obtain the object nodes as “Object 2” and “Object 3”, and perform weighted sum of the fraud risk values of “Object 2” and “Object 3” to obtain the related information of risk person of the assistance applicant based on the node of the assistance applicant.

In some embodiments, before the social assistance management platform obtains an object node connected to the edge that the adjacency degree is larger than 1, the maximum adjacency degree may be set in advance to clarify the related range. The maximum adjacency degree may be determined based on the experience. For example, the maximum adjacency degree is preset to 5, and the social assistance management platform obtains the object nodes connected to the edges that the adjacency degree is less than or equal to 5 and the type is “direct relatives” or “the same address”, and perform weighted sum on the fraud risk values of the plurality of the found object nodes to obtain related information of risk person of the assistance applicant.

It may also be understood that the living environment can reflect the level of fraud risk in a certain extent. For example, people living in the slums may have a higher fraud risk than those living in upscale neighborhoods. It may also be understood that relatives may also have an impact on whether a person performs a fraud. For example, if parents are honest and trustworthy, the fraud risk of their children is very little. Therefore, performing a comprehensive evaluation of the assistance applicant based on the fraud risk values of the related person of the assistance applicant can further enhance the accuracy of the assessment.

In some embodiments, the weight of the information obtained based on the nodes connected to the edges of the “direct relatives” type may be larger than the weight of the information obtained based on the nodes connected to the edges of the “same address” type. For example, if the edge of “Object A” that the adjacency degree is 1 and the type is the “direct relatives” in the integrated information knowledge graph is connected to “Object B”, the edge that the adjacency degree is 1 and the type is the “same address” is connected to “Object C”, and “Object B” and “Object C” are not the same person; then in a calculation, the weight of the information obtained based on “Object B” is larger than the weight of the information obtained based on “Object C”. For example, the weight of the information obtained based on “Object B” is 0.7, and the weight of the information obtained based on “Object C” is 0.3.

In some embodiments, the weight of the information is smaller if it is obtained based on the node connected to the edge with a larger adjacency degree. For example, if the edge of “Object A” that the adjacency degree is 1 in the integrated information knowledge graph is connected to “Object B”, the edge that the adjacency degree is 2 is connected to “Object C”, then in a calculation, the weight of the information obtained based on “Object B” is larger than the weight of the information obtained based on “Object C”. For example, the weight of the information obtained based on “Object B” is 0.7, and the weight of the information obtained based on “Object C” is 0.3.

In some embodiments, the social assistance management platform may inquire attributes of the edge, that adjacency degree is 1 and the type is “assistance application”, of nodes corresponding to the assistance applicant from the assistance applicant from the integrated information knowledge graph, so as to obtain the personal historical social assistance application information of the assistance applicant. For example, the social assistance management platform may find the node corresponding to the assistance applicant and determine the attributes of the edge that the adjacency degree is 1 and the type is the “assistance application” as the personal historical social assistance application information of the assistance applicant, such as the count of times of application, the amount of the application, the total application amount, etc. As shown in FIG. 6, the personal historical social assistance application information of object 1 may be obtained based on the attributes of the edge between “Object 1” and “First Social Assistance Institution”, which includes that the count of times of application is 6, the amount of the application is 5,000 yuan, and the total application amount is 30,000 yuan, etc.

In some embodiments, when there are multiple edges of the node corresponding to the assistance applicant that the adjacency degree is 1 and the type is the “assistance application” in the integrated information knowledge graph, the social assistance management platform may use methods such as performing a sum on the feature related to the assistance application information in the attribute information corresponding to the multiple edges to obtain the personal historical social assistance application information of the assistance applicant.

In some embodiments, the social assistance management platform may, starting from the node corresponding to the assistance applicant in the integrated information knowledge graph, obtain object nodes connected to the edges that the adjacency degree is larger than or equal to 1, find the attribute information of the edges that are connected to these object nodes and the type of that is the “assistance application”, and perform weighted sum on the features related to the assistance application information to obtain the historical social assistance application information of the related person of the assistance applicant.

In some embodiments, when the social assistance management platform obtains an object node connected to the edge that the adjacency degree is larger than 1, the maximum adjacency degree may be set in advance. For example, the maximum adjacency degree is preset to 3.

Step 630, obtain fraud risk information of the assistance object by performing a fraud risk assessment on the assistance object based on the auxiliary risk assessment information.

Fraud risk information may refer to the possibility of the assistance applicant performing a fraud on the assistance application. Fraud risk information may include having a fraud risk, no fraud risk, a fraud risk value, etc. Fraud risk information may have a variety of expression methods such as numbers and words, for example, having a fraud risk, the fraud risk value being 0.8, etc. The fraud risk value may be represented by the number between 0 and 1, the larger the value, the more likely for the assistance applicant to perform fraud.

In some embodiments, the social assistance management platform may compare the fraud risk value with the preset threshold to obtain the fraud risk information of the assistance object based on the auxiliary risk assessment information. For example, the preset standard in step 330 may include the preset threshold, such as, the preset standard may include that the deposit amount is less than 10,000 yuan, the count of the serious disease is larger than 3, the count of times of receiving free food is less than 10, etc. If one or more information in the auxiliary risk assessment information does not meet the preset standard, the social assistance management platform may determine the fraud risk information of the assistance object as having a fraud risk.

In some embodiments, the social assistance management platform may determine the fraud risk value of the assistance object by processing auxiliary risk assessment information based on a risk prediction model.

For the description of the auxiliary risk assessment information, see the relevant description in the above step 620.

The risk prediction model may refer to a machine-learning model after training. In some embodiments, the risk prediction model may be a deep neural network model. In some embodiments, the risk prediction model may include other models. For example, a recurrent neural network model, a convolutional neural network or other customed model structure, or the like, or any combination thereof.

In some embodiments, multiple training samples with a label may be used to train the risk prediction model through multiple methods (such as a gradient drop method) to learn the parameters of the model. When the training model meets the preset condition, the training ends and the risk prediction model having been trained is obtained.

Training samples may include information related to person nodes in the historical integrated information knowledge graph, for example, historical auxiliary risk assessment information. Historical auxiliary risk assessment information may include historical credit information, historical personal medical information, historical related person information, and historical count of times of receiving free food, etc. For the description of the historical auxiliary risk assessment information, see the above-mentioned description of auxiliary risk assessment information. The label of the training sample may be the historical fraud risk value. The label of a training sample may be obtained by manual labeling. For example, the label of the person with a fraudulent assistance behavior (or multiple fraudulent assistance behaviors, because a single fraudulent assistance behavior may be misjudgment by the model) is set to 1, and the label of the person who obviously does not have a fraudulent assistance behavior (may be evaluated manually) is set to 0. In some embodiments, the risk prediction model may be trained in other devices or modules.

In some embodiments, the social assistance management platform may assign the fraud risk value of the assistance object to the fraud risk value in the node attribute of the assistance object. Such setting can facilitate to judge the fraud risks of other objects by using the assistance object.

In some embodiments, the social assistance management platform may evaluate the assistance object with multiple times based on other information. For example, the social assistance management platform may evaluate the assistance object again based on the clothes worn by the assistance object and the mobile phone used by the assistance object.

It may be understood that the travel tools of most people who need to be assisted are basically public vehicles, such as buses and subways. Therefore, other information may include bus-riding information.

In some embodiments, the process 600 may also include: determining a re-assessment assistance object based on the fraud risk information; obtaining bus-riding information of the re-assessment assistance object based on a bus system; and adjusting the fraud risk information of the re-assessment assistance object based on the bus-riding information.

The re-assessment assistance object may be the assistance object that has been evaluated, for example, the assistance object having passed an evaluation. In some embodiments, the re-assessment assistance object may include the assistance object who has passed an evaluation, but the fraud risk value is higher.

In some embodiments, the social assistance management platform may determine the assistance object with the fraud risk value exceeding a threshold as a re-assessment assistance object. For example, the social assistance management platform may determine the assistance object with a fraud risk value exceeding 60% of the threshold as a re-assessment assistance object.

In some embodiments, the social assistance management platform may obtain the fraud risk value through the attributes of the object node. For example, the assistance management platform may obtain the fraud risk value of the node “Object 1” based on the fraud risk value included in the attribute of the node “Object 1”.

In some embodiments, the social assistance management platform may obtain the fraud risk value through the output of the risk prediction model.

Bus-riding information may refer to information related to riding a bus. For example, the station of getting on a bus, the station of getting off a bus, the count of times of riding a bus, etc.

In some embodiments, the social assistance management platform may obtain the bus-riding information of the re-assessment assistance object by querying the bus system based on the identity information of the re-assessment assistance object.

In some embodiments, the social assistance management platform may adjust the fraud risk information of the re-assessment assistance object based on the bus-riding information.

In some embodiments, the social assistance management platform may reduce the fraud risk value in the fraud risk information of the re-assessment assistance object when the count of riding a bus of the re-assessment assistance object is larger. In some embodiments, the social assistance management platform may reduce the fraud risk value in the fraud risk information of the re-assessment assistance object in different degrees based on regional information, the start location and end location of riding the bus. For example, the private lanes in a certain area are blocked and the bus lanes are spacious, so many people choose to travel by bus, so people who often take a bus in this area may not be the person who needs to be assisted. Therefore, the fraud risk value of the re-assessment assistance object in this region may be decreased.

In some embodiments, the social assistance management platform may assign the fraud risk value after being adjusted in the fraud risk information of the assistance object to the fraud risk value in the attribute of the object node corresponding to the assistance object in the integrated information knowledge graph. Such setting can facilitate to judge the fraud risks of other objects by using the assistance object.

Some embodiments of the present disclosure evaluate the assistance object again based on bus-riding information. The feature considered is more comprehensive and the accuracy of the evaluation can be improved.

In some embodiments, the social assistance management platform may adjust the fraud risk information of the re-assessment assistance object based on bus-riding information in combination with other information, for example, the information of the fellow person of the re-assessment assistance object, the bus line of the re-assessment assistance object often riding, etc.

In some embodiments, the social assistance management platform may adjust the fraud risk information of the re-assessment assistance object based on bus-riding information and positioning information of the re-assessment assistance object. For example, the positioning information of the re-assessment assistance object indicates that he/she moves rapidly, but the bus-riding information cannot be inquired, indicating that the person is likely to take a private car or a taxi. The social assistance management platform may improve the fraud risk value in the fraud risk information of the person.

Positioning information may refer to the geographical location of the assistance object, for example, the real-time movement trajectory of the assistance object on the map. In some embodiments, the social assistance management platform may obtain the positioning information based on the device carried with the re-assessment assistance object that may be used to position, such as a phone.

In some embodiments, the social assistance management platform may assign the fraud risk value after being adjusted in the fraud risk information of the assistance object to the fraud risk value in the attribute of the object node corresponding to the assistance object in the integrated information knowledge graph. Such setting can facilitate to judge the fraud risks of other objects by using the assistance object.

Some embodiments of the present disclosure evaluate the assistance object again based on not only bus-riding information but also positioning information. The feature considered is more comprehensive and the accuracy of the evaluation can be improved.

Some embodiments of the present disclosure can effectively identify the fraud risk of the target person by using the integrated information knowledge graph and the risk prediction model based on the massive data provided by the knowledge graph and the high accuracy determination of the machine learning model, which helps the social assistance institution to perform precise assistance.

It should be noted that the description of the above-mentioned process 600 is only for examples and descriptions, but does not limit the scope of the application of the present disclosure. For those skilled in the art, various modifications and changes may be made to process 600 under the guidance of the present disclosure. However, these modifications and changes are still within the scope of the present disclosure.

The basic concepts have been described above, apparently, for those skilled in the art, the above-mentioned detailed disclosure is only used as an example, and it does not constitute a limitation of the present disclosure. Although there is no clear explanation here, those skilled in the art may make various modifications, improvements, and corrections for the present disclosure. Such modifications, improvements, and corrections are suggested in the present disclosure, so such modifications, improvements, and corrections still belong to the spirit and scope of the exemplary embodiments of the present disclosure.

Meanwhile, the present disclosure uses specific words to describe the embodiments of the present disclosure. For example, “one embodiment,” “an embodiment,” and/or “some embodiments” mean that a certain feature, structure, or characteristic is connected with at least one embodiment of the present disclosure. Therefore, it should be emphasized and noted that two or more references of “an embodiment” or “one embodiment” or “an alternative embodiment” in various places in the present disclosure do not necessarily refer to the same embodiment. In addition, some characteristics, structures, or characteristics of one or more embodiments in this manual may be properly combined.

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose of description and that the appended claims are not limited to the disclosed embodiments, on the contrary, are intended to cover modifications and equivalent combinations that are within the spirit and scope of the embodiments of the present disclosure. For example, although the implementation of various components described above may be embodied in a hardware device, it may also be implemented as a software only solution, e.g., an installation on an existing server or mobile device.

Similarly, it should be noted that to simplify the expressions disclosed in the present disclosure and thus help the understanding of one or more embodiments of the invention, in the foregoing description of the embodiments of this specification, various features may sometimes be combined into one embodiment, drawings or descriptions thereof. However, this disclosure method does not mean that the characteristics required by the object of the present disclosure are more than the characteristics mentioned in the claims. Rather, claimed subject matter may lie in less than all features of a single foregoing disclosed embodiment.

Some embodiments use numbers with description ingredients and attributes. It should be understood that the number described by such examples is used in some examples with the modified words “about”, “approximate” or “generally” to modify. Unless otherwise stated, “about”, “approximate” or “generally” indicates that the number allows a change of ±20%. Correspondingly, in some embodiments, the value parameters used in the present disclosure and claims are approximate values. The approximate values may be changed according to the characteristics of individual embodiments. In some embodiments, the numerical parameters should consider the effective digits specified and use a general digit reservation method. Although in some embodiments of the present disclosure, the numerical domain and parameters used to confirm the range of its scope are approximate values, the setting of such values may be as precise as possible within the feasible range in specific embodiments.

For each patent, patent application, patent application publications and other materials cited by the present disclosure, such as articles, books, instructions, publications, documents, etc., all of them will be incorporated in the present disclosure as a reference. History application documents that are inconsistent or conflictive with the contents of the present disclosure are excluded, as well as documents (currently or subsequently appended to the present disclosure) limiting the broadest scope of the claims of the present specification. It should be noted that, if there is any inconsistency or conflict between the descriptions, definitions, and/or usage of terms in subsidiary information of the present disclosure and the contents of the present disclosure, the descriptions, definitions, and/or usage of terms in the present disclosure shall prevail.

Finally, it should be understood that the embodiments described in the present disclosure are only used to illustrate the principle of the embodiments of the present disclosure. Other deformations are also possible within the scope of the present disclosure. Therefore, merely by way of example and not limitation, alternative configurations of the embodiments of the present disclosure may be considered consistent with the teachings of the present disclosure. Correspondingly, the embodiments of the present disclosure are not limited to the embodiments clearly introduced and described in the present disclosure.

What is claimed is:

1. A method for social assistance review of a smart city based on Internet of Things, wherein the method is performed by a social assistance management platform, the method comprising:
obtaining a social assistance application of an assistance applicant based on a user platform by a social assistance service platform;
obtaining auxiliary reviewing information; and
determining an assistance object passing the review by reviewing the social assistance application based on the auxiliary reviewing information.

2. The method of claim 1, wherein the obtaining auxiliary reviewing information comprises:
sending amount limit information of deposit and amount limit information of consumption to a financial management system; and
obtaining a first feature of the assistance applicant based on security computing of privacy protection through the financial management system.

3. The method of claim 1, wherein the obtaining auxiliary reviewing information comprises:
obtaining a medical knowledge graph of the assistance applicant based on a medical system; and
obtaining a second feature of the assistance applicant based on the medical knowledge graph.

4. The method of claim 3, wherein the method further comprises sending auxiliary medical information of the assistance object to the medical system.

5. The method of claim 1, wherein the obtaining auxiliary reviewing information comprises:
obtaining a food-received knowledge graph of the assistance applicant based on a food distribution system; and
obtaining a third feature of the assistance applicant based on the food-received knowledge graph.

6. The method of claim 5, wherein the method further comprises sending auxiliary food information of the assistance object to the food distribution system.

7. The method of claim 1, wherein the method further comprises:
obtaining an integrated information knowledge graph of the assistance applicant based on a social assistance system;
obtaining at least one of credit information of the assistance applicant, financial information of the assistance applicant, medical information of the assistance applicant, medical information of a related person of the assistance applicant, free food-received information of the assistance applicant, related information of a risk person of the assistance applicant, historical social assistance application information of the assistance applicant, historical social assistance application information of the related person of the assistance applicant as auxiliary risk assessment information based on the integrated information knowledge graph; and
obtaining fraud risk information of the assistance object by performing a fraud risk assessment on the assistance object based on the auxiliary risk assessment information.

8. The method of claim 7, wherein the method further comprises:
determining a re-assessment assistance object based on the fraud risk information;
obtaining bus-riding information of the re-assessment assistance object based on a bus system; and
adjusting fraud risk information of the re-assessment assistance object based on the bus-riding information.

9. The method of claim 8, wherein the adjusting the fraud risk information of the re-assessment assistance object based on the bus-riding information comprises:
adjusting the fraud risk information of the re-assessment assistance object based on the bus-riding information and positioning information of the re-assessment assistance object.

10. A system for social assistance review of a smart city based on Internet of Things, comprising a user platform, a social assistance service platform, and a social assistance management platform, wherein the social assistance management platform is configured to perform the following operations comprising:
obtaining a social assistance application of an assistance applicant based on the user platform by the social assistance service platform;
obtaining auxiliary reviewing information; and
determining an assistance object passing the review by reviewing the social assistance application based on the auxiliary reviewing information.

11. The system of claim 10, wherein to obtain the auxiliary reviewing information, the social assistance management platform is further configured to perform the following operations including:
sending amount limit information of deposit and amount limit information of consumption to a financial management system; and
obtaining a first feature of the assistance applicant based on security computing of privacy protection through the financial management system.

12. The system of claim 10, wherein to obtain the auxiliary reviewing information, the social assistance management platform is further configured to perform the following operations including:
obtaining a medical knowledge graph of the assistance applicant based on a medical system; and
obtaining a second feature of the assistance applicant based on the medical knowledge graph.

13. The system of claim 12, wherein the social assistance management platform is further configured to perform the following operations including:
sending auxiliary medical information of the assistance object to the medical system.

14. The system of claim 10, wherein the social assistance management platform is further configured to perform the following operations including:
obtaining a food-received knowledge graph of the assistance applicant based on a food distribution system; and
obtaining a third feature of the assistance applicant based on the food-received knowledge graph.

15. The system of claim 14, wherein the social assistance management platform is further configured to perform the following operations including:
sending auxiliary food information of the assistance object to the food distribution system.

16. The system of claim 10, wherein the social assistance management platform is further configured to perform the following operations including:
obtaining an integrated information knowledge graph of the assistance applicant based on a social assistance system;
obtaining at least one of credit information of the assistance applicant, financial information of the assistance applicant, medical information of the assistance applicant, medical information of a related person of the assistance applicant, free food-received information of the assistance applicant, related information of a risk person of the assistance applicant, historical social assistance application information of the assistance applicant, historical social assistance application information of the related person of the assistance applicant as auxiliary risk assessment information based on the integrated information knowledge graph; and
obtaining fraud risk information of the assistance object by performing a fraud risk assessment on the assistance object based on the auxiliary risk assessment information.

17. The system of claim 16, wherein the social assistance management platform is further configured to perform the following operations including:
determining a re-assessment assistance object based on the fraud risk information;
obtaining bus-riding information of the re-assessment assistance object based on a bus system; and
adjusting the fraud risk information of the re-assessment assistance object based on the bus-riding information.

18. The system of claim 17, wherein the social assistance management platform is further configured to perform the following operations including:
adjusting the fraud risk information of the re-assessment assistance object based on the bus-riding information and positioning information of the re-assessment assistance object.

19. A computer-readable storage medium comprising computer instructions, wherein when reading the computer instructions in the storage medium, the computer executes the method for social assistance review of the smart city based on Internet of Things according to claim 1.
